SEQUENCE LISTING (2 sequences)

SEQ ID NO: 1 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 2 - RNA, 451 nt, Homo sapiens
ggguugcggagggugggccugggaggggugguggccauuuuuugucuaacccuaacugagaagggcguaggcgccgugcuuuugcuccccgcgcgcuguuuuucucgcugacuuucagcgggcggaaaagccucggccugccgccuuccaccguucauucuagagcaaacaaaaaaugucagcugcuggcccguucgccccucccggggaccugcggcgggucgccugcccagcccccgaaccccgccuggaggccgcggucggcccggggcuucuccggaggcacccacugccaccgcgaagaguugggcucugucagccgcgggucucucgggggcgagggcgagguucaggccuuucaggccgcaggaagaggaacggagcgaguccccgcgcgcggcgcgauucccugagcugugggacgugcacccaggacucggcucacacaugc

(19) United States
(12) Patent Application Publication
(10) Pub. No.: US 2009/0142408 A1
(43) Pub. Date: Jun. 4, 2009

(54) Telomerase delivery by biodegradable Nanoparticle

(75) Inventors: Xin Lin, Sacramento, CA (US); Guotang Zhai, Folsom, CA (US); Matthew Sarad, Folsom, CA (US); Pete N. Lohstroh, West Sacramento, CA (US)

Correspondence Address: GUOTANG ZHAI, SUITE 102, 10933 TRADE CENTER DRIVE, RANCHO CORDOVA, CA, 95670, US

(73) Assignee: TELOMOLECULAR CORPORATION, RANCHO CORDOVA, CA (US)

(21) Appl. No.: 12/220,221

(22) Filed: Jul. 22, 2008

Related U.S. Application Data
(60) Provisional application No. 60/951,411, filed on Jul. 23, 2007.

(51) Int. Cl. A61K 9/14; A61K 38/43

(52) U.S. Cl. 424/501; 424/489; 424/94.1

(57) ABSTRACT

A therapeutic compound consisting of human telomerase, its catalytic subunit hTert, or a known variant of either, and a biodegradable nanoparticle carrier, which can be administered to cells in a cell culture or in a living animal, is provided herein. The therapeutic compound is envisioned as a method for treating a wide variety of age-related diseases such as idiopathic pulmonary fibrosis, aplastic anemia, dyskeratosis congenita, arteriosclerosis, macular degeneration, osteoporosis, Alzheimer's, diabetes type 2, and any disease that correlates with telomere shortening and may be corrected or ameliorated by lengthening telomeres. The therapeutic compound is also envisioned as method for potentially treating more generic problems of human aging. The nanoparticle carrier is comprised of certain biodegradable biocompatible polymers such as poly(lactide-co-glycolide), poly(lactic acid), poly(alkylene glycol), polybutylcyanoacrylate, poly(methylmethacrylate-co-methacrylic acid), poly-allylamine, polyanhydride, polyhydroxybutyric acid, polycaprolactone, lactide-caprolactone copolymers, polyhydroxybutyrate, polyalkylcyanoacrylates, polyanhydrides, polyorthoester or a combination thereof. The nanoparticle may incorporate a targeting moiety to direct the nanoparticle to a particular tissue type or a location within a cell. The nanoparticle may incorporate a plasticizer to facilitate sustained release of telomerase such as L-tartaric acid dimethyl ester, triethyl citrate, or glyceryl triacetate. A nanoparticle of the present invention can further contain a polymer that affects the charge or lipophilicity or hydrophilicity of the particle. Any biocompatible hydrophilic polymer can be used for this purpose, including but not limited to, poly(vinyl alcohol).

### CLAIM OF PRIORITY

This application claims priority under 35 U.S.C. §119(e) to U.S. Provisional Application, 60/951,411 filed Jul. 23, 2007, the entire content of which is incorporated herein by reference.

### FIELD OF THE INVENTION

The present invention relates to the field of telomere elongation through functional telomerase enzyme cellular delivery by means of its encapsulation in nanoparticles for treating aging and age-related diseases or conditions. More specifically, the invention provides telomerase enzyme-containing nanoparticles and methods of use thereof for the treatment of diseases associated with lack of sufficient cellular telomerase activities or aberrant telomerase functions, including without limitation, idiopathic pulmonary fibrosis, dyskeratosis congenita, aplastic anemia, arteriosclerosis, macular degeneration, osteoporosis, Alzheimer's, diabetes type 2, and any disease that is either caused by or correlates with telomere shortening and may be corrected or ameliorated by lengthening telomeres.

### BACKGROUND OF THE INVENTION

Several references and patent documents are cited throughout this application to better define the state of the art to which the invention pertains. Each of these citations is incorporated by reference herein as though set forth in full.

Human telomerase reverse transcriptase protein component (hTert) is a catalytic molecular entity that has been shown to spontaneously reconstitute the whole enzyme telomerase in human cells with its RNA component known as hTR or hTERC. In nature, telomerase repairs the ends of chromosomal DNA by elongating chromosomal telomeres. Abnormal or accelerated shortening of telomeres plays a known pathological role in the development of a number of age-associated disease states, such as idiopathic pulmonary fibrosis (Tsakiri et al., Proc Natl Acad Sci USA 2007; 104: 7552-7557; Armanios et al., N Engl J Med 2007; 356: 1317-1326), dyskeratosis congenita (Mitchell et al., Nature 1999; 402: 551-555), aplastic anemia (Brummendorf et al., Ann NY Acad Sci 2001; 938: 293-303), arteriosclerosis (Okuda et al., Atherosclerosis. 2000; 152(2):391-8), osteoporosis (Kveiborg et al., Mech Ageing Dev. 1999; 106(3):261-71), and macular degeneration (Matsunaga et al., Invest Opthalmol Vis Sci. 1999; 40(1):197-202). Normal or natural telomere shortening is also known to play an important role in the general processes of human and animal aging; therefore, it has been postulated by some skilled in the art that telomerase enzyme may hold potential as a therapeutic agent for the correction or amelioration of the pathological conditions such as those indicated above. Development of telomerase-based drugs would represent a novel therapeutic frontier in medicine as it might lead to a new category of pharmaceutical compounds that are for the first time able to effectively treat various incurable diseases or conditions.

In vivo therapeutic delivery of the enzyme telomerase alone was first proposed by Dr. Michael West although no specific instructions on how to accomplish this were provided (West et al., U.S. Pat. No. 5,489,508). Telomerase has been widely speculated as a useful agent in the treatment of disease and has been regarded as a potential “therapeutic protein” (Harley et al., Experimental Gerontology 27:375-382 1992). Moreover, a concept called transient immortalization described the use of hTert in vitro for the growth of cells in a petri dish that could be later safely transplanted into a living organism (Baetge et al., U.S. Pat. No. 6,358,739). It was also suggested that this immortalization concept could be applied to in vivo settings such as human tissues, organs, systems or even the entire body.

While the prospect of harnessing telomerase delivery to treat diseases or conditions remains fascinating, practical aspects are principally restricted by the limitations of existing gene delivery technologies that are not capable of efficiently and safely delivering enzyme genes of such a large size to across cellular or subcellular membranes that protect cells from protein escape or penetration (Labhasetwar et al., The FASEB Journal. 2002; 16:1217-1226). A number of vectors used in gene therapy including viruses, fusogen peptides, cationic lipids, and cationic polymers have been shown to deliver molecules into the cytosol of a cell. However, these carriers suffer from a number of limitations including immunogenicity, toxicity, instability in vivo, and the ability to deliver molecules of limited size and weight (Maheshwari, Mol. Ther. 2000; 2, 121-130). Protein transduction domains (PTDs) and some cell penetrating peptides have been demonstrated to carry large molecular payloads such as proteins across biological membranes into the cytosol of a cell (Lindgren, Trends Pharmacol. Sci. 2000; 21, 99-103). However, these vectors suffer from certain disadvantages in that they require complex engineering to cross-link to a target peptide or protein (Morris et al. Nat. Biotechnol. 2001; 19, 1173-1176) and also, some of these systems derived from HIV-1 TAT protein or an adenovirus, for example, require denaturation of the protein (Schwarze et al., Trends Pharmacol. Sci. 2000; 21, 45-48). More recently, a short amphipathic carrier, Pep-1, was used to deliver functionally active proteins and peptides intracellularly without the need for cross-linking or denaturation. However, particularly sensitive proteins, like telomerase or its protein component hTert, can be damaged as they are crossing the cell membranes if they are left unprotected, which may result in enzymatic inactivity.

A practical method for safely and efficiently delivering telomerase and/or its catalytic subunit hTert into cells is desirable from the standpoint that it might lead to pharmacological innovations that may correct or ameliorate serious age-associated pathologies or conditions. A method for achieving the safe and efficient delivery of this special class of enzymes could be through the use of biodegradable nanoparticles. The rapid (<10 min) endo-lysosomal escape of biodegradable nanoparticles formulated from the copolymers of poly(D,L-lactide-co-glycolide) (PLGA) has been noted when delivering large and sensitive enzymes to cells (Labhasetwar et al., The FASEB Journal 2002; 16:1217-1226). The mechanism of rapid escape is by selective reversal of the surface charge of nanoparticle (from anionic to cationic) in the acidic endo-lysosomal compartment, which causes the nanoparticle to interact with the endo-lysosomal membrane and escape into the cytosol. Diffusion of the nanoparticles can cause materials to pass through the nuclear pore and enter the nucleus where the chromosomal telomeres are located.

Biodegradable nanoparticles have been shown to be capable of delivering a variety of therapeutic agents including macromolecules such as proteins (Labhasetwar et al., The FASEB Journal. 2002; 16:1217-1226) and low molecular weight drugs such as dexamethasone, intracellularly at a slow rate, which results in a sustained therapeutic effect (Guzman et al., Circulation 1996; 94:1441-1448). Conversely, the payload can also be engineered to release rapidly. While the use of cell penetrating peptides such as VP22 has been described as an approach for delivering hTert gene in vitro (U.S. Pat. No. 6,358,739), these carriers have little in common with nanoparticle formulations and do not enable the use of nanoparticles as a carrier. PLGA has a number of advantages over other polymers used in drug and gene delivery including biodegradability, biocompatibility, and approval for human use granted by the U.S. Food and Drug Administration. PLGA has been studied extensively and is considered an apposite means for sustained intracellular delivery of macromolecules (Panyam & Labhasetwar, Molecular Pharmaceutics 2004; 1:77-84).

The entrapment of proteins into nanoparticles has not become a simple or reproducible scientific process and the delivery of proteins often necessitates extensive research and optimizations since each protein is characterized by molecular weight, hydrophilicity, stability. This situation often complicates protein-based therapeutic formulation. Inherently, each new class of proteins to be tested in this format requires specific reduction to practice to both prove feasibility and to develop workable formulations. Whether a protein represents a new class depends greatly on its dimension. The choice of a correct formulation strategy is considerably determined by protein solubility, size, and molecular stability. With 1,132 amino acids and a molecular weight of 126,997 Daltons, the hTert protein is exceptionally large from a biomolecular standpoint, while the full human telomerase enzyme nearly doubles in weight. The molecular size and weight of these enzymes are much larger than other enzymes (known to the inventors) that have been delivered with a nanoparticle formulation. Telomerase is generally considered unstable (Koo, United States Patent Application 20030148988), therefore it has been unknown whether it could be successfully encapsulated and delivered to cells in a functional state via nanoparticles. Because of its instability, particularly in light of the need to cross cell membranes, it is not enough of a scientific undertaking to simply deliver telomerase (or its subunit) into a cell regardless of its function; it must be demonstrated to function upon delivery by being able to elongate human chromosomal telomeres. Any study that fails to show post-delivery enzymatic activity should be considered incomplete. In the following study we demonstrate a method and composition for encapsulating human telomerase, both hTert subunit and hTR subunit, and variants thereof in a biodegradable nanoparticle that can then be delivered to cells in cell culture or in a living animal. As the nanoparticle degrades it releases telomerase in a therapeutically sustained way and successfully elongates chromosomal telomeres.

U.S. patent application Ser. No. 09/847,945 teaches methods for treating hyperplasia in a subject by delivering at least one drug in nanoparticle form and dispersed in a biocompatible protein. This reference discloses the use of paclitaxel, rapamycin, steroids, and the like, as suitable candidates to inhibit proliferation and migration of cells. This reference does not teach block co-polymer nanoparticles.

U.S. Pat. No. 6,322,817 teaches a pharmaceutical formulation of paclitaxel, wherein the paclitaxel is entrapped into nanoparticles comprising at least one type of amphiphilic monomer which is polymerized by adding an aqueous solution of cross-linking agent. This reference discloses a preferred combination of amphiphilic monomers comprising vinyl pyrrolidone, N-isopropylacrylamide, and monoester of polyethylene glycol maleic anhydride cross-linked with a bi-functional vinyl derivative such as N,N′-methylene bis-acrylamide useful in the treatment of pathological conditions arising out of excessive proliferation of cells such as rheumatoid arthritis or cancer.

U.S. Pat. No. 6,759,431 discloses methods for treating or preventing diseases associated with body passageways by delivering to an external portion of the body passageway a therapeutic agent such as paclitaxel, or an analogue or derivative thereof encapsulated in polymeric carriers.

U.S. Pat. No. 7,332,159 discloses methods for preventing reperfusion injury following stroke by delivering antioxidants in a sustained release biodegradable nanoparticle.

U.S. Pat. No. 6,358,739 discloses methods for transiently immortalizing a cell with an effective dose of human telomerase reverse transcriptase gene. This reference does not teach the use of biodegradable nanoparticles as a carrier and this reference does not teach a method for the efficient in vivo delivery of a therapeutic compound.

U.S. Pat. No. 5,583,016 discloses methods and procedures for identifying and producing wild-type telomerase and its catalytic subunit hTert. This reference does not teach a method for the effective in vivo delivery of this enzyme for the treatment of a disease or condition.

U.S. Pat. No. 6,093,809 discloses methods for the delivery of genes to cells that cause continuous production of telomerase. This reference does not teach the delivery of a protein.

### SUMMARY OF THE INVENTION

The present invention relates to a method and a composition for administering an effective amount of telomerase, its subunit hTert, or a known variant of either, wherein said agent is formulated in a nanoparticle and is administered orally, topically, intravenously through a normal route, or in the instance of application to the brain intrathecally or intravenously via the carotid artery or jugular vein to a subject in need of treatment, thereby treating a specific disease or condition that is caused by or correlates with telomere shortening, including but not limited to: idiopathic pulmonary fibrosis (Tsakiri et al., Proc Natl Acad Sci USA 2007; 104: 7552-7557; Armanios et al., N Engl J Med 2007; 356: 1317-1326), dyskeratosis congenita (Mitchell et al., Nature 1999; 402: 551-555), aplastic anemia (Brummendorf et al., Ann NY Acad Sci 2001; 938: 293-303), arteriosclerosis (Okuda et al., Atherosclerosis. 2000; 152(2):391-8), osteoporosis (Kveiborg et al., Mech Ageing Dev. 1999; 106(3):261-71), and macular degeneration (Matsunaga et al., Invest Opthalmol Vis Sci. 1999; 40(1):197-202), cirrhosis of the liver (Kitada et al., Biochem Biophys Res Commun. 1995; 211(1):33-9, arthritis (Salmon et al., Trends Immunol. 2004; 25(7):339-41), Alzheimer's (Thomas et al., Mech Ageing Dev. 2008; 129(4):183-90), diabetes (Sampson et al., Diabetes Care. 2006; 29(2):283-9), wrinkling of the skin (Allsopp et al., Proc Natl Acad Sci USA. 1992; 89(21):10114-8), graying of hairs (Chang et al., Nat. Genet. 2004; 36(8):877-82), and any disease of aging that can be treated in this way (Blasco et al., Nat Chem. Biol. 2007; 3(10):640-9). In certain embodiments, the enzyme to be administered is the entire human telomerase holoenzyme (hTert and hTR), in others it is hTert subunit only, while in others it may be a combination thereof or closely related variants of either enzyme component that perform the same function. In the preferred embodiment the nanoparticle is composed principally of a biodegradable polymer such as poly(lactide-co-glycolide), poly(lactic acid), poly(alkylene glycol), polybutylcyanoacrylate, poly(methylmethacrylate-co-methacrylic acid), poly-allylamine, polyanhydride, polyhydroxybutyric acid, polycaprolactone, lactide-caprolactone copolymers, polyhydroxybutyrate, polyalkylcyanoacrylates, polyanhydrides, polyorthoester or a combination thereof. In still further embodiments, the nanoparticle contains a targeting moiety or a plasticizer such as L-tartaric acid dimethyl ester, triethyl citrate, or glyceryl triacetate to facilitate sustained release of telomerase. A nanoparticle can be said to have core ingredients that facilitate the transduction process causing the nanoparticle to cross the cellular membrane. Compounds that are not considered core ingredients can be added to the nanoparticle to change the profile of its release, targeting, and localization in or to a cell. A plasticizer can be added to change the nature or sustainability of the protein release. A list of satisfactory plasticizers (in addition to those mentioned above) is described in this document. A targeting moiety can be added to the nanoparticle that increases cellular uptake efficiency, targets the nanoparticle to a specific cell, or localizes the nanoparticle somewhere within a cell. The process of attaching such moieties is generic in nature. Other ingredients, particularly biodegradable or biocompatible ingredients, can be added that do not necessarily change the net effect of the nanoparticle. If these other ingredients do not serve an essential role they are considered superfluous to the formulation.

It has been shown in other studies (Labhasetwar et al., U.S. Pat. No. 7,332,159) that the nanoparticles can efficiently cross the blood brain barrier and treat conditions of the brain by delivering therapeutic proteins such as superoxide dismutase, catalase, glutathione peroxidase, glutathione reductase, glutathione-S-transferase hemeoxygenase, or mimetic or synthetic enzymes thereof. It has further been demonstrated that when the nanoparticle formulation contains a plasticizer such as dimethyl tartrate (DMT), sustained “controlled” release of the active agent can be achieved.

### BRIEF DESCRIPTION OF THE DRAWINGS

FIG. 1: Telomere elongation in living human cells by human telomerase 24 hours following nanoparticles cellular delivery.

FIG. 2: Telomere elongation in living human cells by human telomerase 48 hours following nanoparticles cellular delivery.

### DEFINITIONS

The following definitions are provided to facilitate an understanding of the present invention:

The terms “telomerase”, “hTert”, or “telomerase protein component”, or “hTR”, or “hTERC” all refers to the essential structural entity of the nuclear enzyme that plays a role in the protection and maintenance of human or animal chromosomal telomeres. It is an enzyme that adds specific DNA sequence repeats (“TTAGGG” in all vertebrates) to the 3′ (“three prime”) end of DNA strands in the telomere regions located at the ends of eukaryotic chromosomes. The telomeres give rise to stability to the chromosomes. The enzyme is a reverse transcriptase that carries its own RNA molecule, which is used as a template when it elongates telomeres, which are shortened after each replication cycle. In adults, telomerase is highly expressed in cells that need to divide regularly (e.g., in the immune system), whereas most somatic cells express it only at very low levels in a cell-cycle dependent manner. While it is currently unknown to what extent telomere erosion contributes to the normal aging process, maintenance of DNA in general and telomeric DNA specifically, have emerged as major therapeutic frontiers.

The term “nanoparticle” refers to a particle having a size measured on the nanometer scale. As used herein, the “nanoparticle” refers to a particle having a matrix-type structure with a size of less than about 1,000 nanometers. When the nanoparticle includes a bioactive component, the bioactive component is entangled or embedded in the matrix-type structure of the nanoparticle. Nanoparticles include particles capable of containing a therapeutic/diagnostic agent that is to be released within a mammalian body, including specialized forms such as nanospheres, whether natural or artificial.

The term “delivery” as used herein refers to the introduction of foreign molecule (i.e., protein containing nanoparticle) in cells.

The term “treating” as used herein means the prevention, reduction, partial or complete alleviation or cure of a disease.

The term “administration” as used herein means the introduction of a foreign molecule (i.e., protein containing nanoparticle) into a cell. The term is intended to be synonymous with the term “delivery”. Administration also refers to the methods of delivery of the compounds of the invention (e.g., routes of administration such as, without limitation, intravenous, intra-arterial, intramuscular, subcutaneous, intrasynovial, infusion, sublingual, transdermal, oral, or topical). The preferred method of delivery is to the blood vessel (e.g., artery or vein) or in particular applications to the carotid, coronary, femoral, renal, or cerebral artery, depending on the site of injury.

As used herein, an “effective amount” of the telomerase or telomerase variants is an amount sufficient to cause telomere elongation, sufficient to make a detectable difference in cellular metabolism, or an amount that may address a disease or condition of aging, in a subject.

An “individual” as used herein refers to any vertebrate animal, preferably a mammal, and more preferably a human.

### DETAILED DESCRIPTION OF THE INVENTION

As one of skill in the art will appreciate, a nanoparticle in accordance with the methods and compositions of the present invention can be composed of a variety of injectable biodegradable polymers. Nanoparticles are said to be biodegradable if the polymer of the nanoparticle dissolves or degrades within a period that is acceptable in the desired application (usually in vivo therapy), usually less than five years, and desirably less than one year, upon exposure to a physiological solution of pH 6-8 having a temperature of between 25° C. and 37° C. As such, a nanoparticle for use in accordance with the methods and compositions of the present invention can be composed homopolymers or copolymers prepared from monomers of polymers disclosed herein, wherein the copolymer can be of diblock, triblock, or multiblock structure. Suitable polymers include, but are not limited to, poly(lactide-co-glycolide), poly(lactic acid), poly(alkylene glycol), polybutylcyanoacrylate, poly(methylmethacrylate-co-methacrylic acid), poly-allylamine, polyanhydride, polyhydroxybutyric acid, polycaprolactone, lactide-caprolactone copolymers, polyhydroxybutyrate, polyalkylcyanoacrylates, polyanhydrides, polyorthoester or a combination of these ingredients. In particular embodiments, a nanoparticle is composed of a copolymer of a poly(lactic acid) and a poly(lactide-co-glycolide). Particular combinations and ratios of polymers are well-known to the skilled artisan and any suitable combination can be used in the nanoparticle formulations of the present invention. Generally, the resulting nanoparticle typically ranges in size from between 1 nm and 1000 nm, or more desirably between 1 nm and 100 nm (Labhasetwar et al., U.S. Pat. No. 7,332,159). Keeping the nanoparticles below 100 nm is essential since an inflammatory response has been associated with PLGA nanoparticles of a larger size (Guzman et al., Circulation 1996; 94, 1441-1448).

A nanoparticle of the present invention can further contain a polymer that affects the charge or lipophilicity or hydrophilicity of the particle (Labhasetwar et al., U.S. Pat. No. 7,332,159). Any biocompatible hydrophilic polymer can be used for this purpose, including but not limited to, poly(vinyl alcohol). This modification can play an important role in the efficiency of delivery since collection of nanoparticles in the liver is a common problem.

To further enhance delivery of a therapeutically effective amount of telomerase, a nanoparticle of the present invention can further contain a targeting moiety (e.g. a protein transduction domain) ((Labhasetwar et al., U.S. Pat. No. 7,332,159). As used herein, a targeting moiety is any molecule which can be operably attached to a nanoparticle of the present invention to facilitate, enhance, or increase the transport of the nanoparticle into target tissue. Such a moiety can be a protein, peptide or small molecule. For example, a variety of protein transduction domains, including the HIV-1 Tat transcription factor, Drosophila Antennapedia transcription factor, as well as the herpes simplex virus VP22 protein have been shown to facilitate transport of proteins into the cell (Wadia & Dowdy Curr. Opin. Biotechnol. 2002; 13:52-56). Further, an arginine-rich peptide (Futaki Int. J. Pharm. 2002; 245:1-7), a polylysine peptide containing Tat PTD (Hashida et al., Br. J. Cancer 2004; 90(6):1252-8), Pep-1 (Deshayes et al., Biochemistry 2004; 43(6):1449-57) or an HSP70 protein or fragment thereof (WO 00/31113) is suitable for targeting a nanoparticle of the present invention. Not to be bound by theory, it is believed that such transport domains are highly basic and appear to interact strongly with the plasma membrane and subsequently enter cells via endocytosis (Wadia et al., Nat. Med. 2004; 10:310-315). Animal model studies indicate that chimeric proteins containing a protein transduction domain fused to a full-length protein or inhibitory peptide can protect against ischemic brain injury and neuronal apoptosis, attenuate hypertension, prevent acute inflammatory responses, and regulate long-term spatial memory responses (Blum & Dash Learn. Mem. 2004: 11:239-243; May et al., Science 2000; 289:1550-1554; Rey et al., Circ. Res. 2001; 89:408-414; Denicourt & Dowdy Trends Pharmacol. Sci. 2003; 24:216-218). Nanoparticles may be modified to target a particular tissue type or precancerous cells for example through the use of such targeting moieties. In this case there is a particular need for site-specific therapeutics to prevent the localized pathophysiologic process of select diseases of aging such as macular degeneration of the eyes, cirrhosis of the liver and kidneys, arteriosclerosis of human arteries and diseases of aging in the skins. It may be important from a safety study standpoint to contain the therapeutic agent largely to the type of tissue that is diseased so that experimental risks are contained. The present invention meets this need.

TABLE 1

Exemplary peptide-based targeting moieties

| | | SEQ |
|---|---|---|
| Source | AMINO ACID SEQUENCE | ID NO: |
| PTD-4^{a} | YARAAARQARA | 1 |
| HIV TAT^{a} | YGRKKRRQRRR | 2 |
| PTD-3^{a} | YARKARRQARR | 3 |
| PTD-5^{a} | YARAARRAARR | 4 |
| PTD-6^{a} | YARAARRAARA | 5 |
| PTD-7^{a} | YARRRRRRRRR | 6 |
| ANTp^{b} | RQIKIWFQNRRMKWKK | 7 |
| Transportin^{b} | GWTLNSAGYLLGKINLKALAALAKKIL | 8 |

^{a}Ho, et al., Cancer Res.2001; 61:474.
^{b}Schwartź & Zhang Curr. Opin. Mol. Ther. 2000; 2:2.

Description of the Table: The table above describes the following ligands: PTD-4-a, HIV TATa, PTD-3a, PTD-5a, PTD-6a, PTD-7a, ANTpb, Transportin and their corresponding amino acid sequences: YARAAARQARA, YGRKKRRQRRR, YARKARRQARR, YARAARRAARR, YARAARRAARA, YARRRRRRRRR, RQIKIWFQNRRMKWKK, GWTLNSAGYLLGKINLKALAALAKKIL.

Suitable small molecules targeting moieties which can be attached to a nanoparticle of the present invention include, but are not limited to, nonpeptidic polyguanidylated dendritic structures (Chung et al., Biopolymers 2004; 76(1):83-96) or poly(N-(2-hydroxypropyl)methacrylamide) (Christie et al., Biomed. Sci. Instrum. 2004; 40:136-41). A targeting moiety might also be able to direct the nanoparticle into particular tissue type such as the liver or the brain.

To conjugate or attach the targeting moiety to a nanoparticle of the present invention, standard methods such as the epoxy activation method can be employed. The nanoparticle surface is contacted with an epoxy compound (e.g., Denacol®, Nagase America Co., CA) which reacts with the hydroxyl functional group of, e.g., the PVA associated with the nanoparticle surface. The epoxy activation of the nanoparticle creates multiple sites for reaction with a ligand and also serves as a linkage between the nanoparticle surface and the peptide to avoid steric hindrance for interaction of the peptide with the cell membrane (Labhasetwar et al., J. Pharm. Sci. 1998; 87:1229-34). The epoxy groups can react with many functional groups including amine, hydroxyl, carboxyl, aldehyde, and amide under suitable pH and buffer conditions, therefore increasing the number of possible targeting moieties which can be employed. While this approach has been shown to be feasible in liposomal carriers, it is not so obvious in the use of nanoparticles since there was no report in this regard.

A nanoparticle formulation of the present invention can further contain a plasticizer to facilitate sustained release of the encapsulated active agent by maintaining the structure of the nanoparticle. Release of molecules (e.g. proteins, DNA oligonucleotides) from nanoparticles formulated from block copolymers is, in general, not continuous. Typically, there is an initial release followed by a very slow and insignificant release thereafter. Not to be bound by theory, it is contemplated that the release profile may be as a result of the rapid initial drop in the molecular weight of the polymer which reduces the glass transition temperature of the polymer to below body temperature (37° C.); the glass transition temperature of copolymers prior to release is above body temperature (˜45° C. to 47° C.). Moreover, with degradation, these polymers become softer thereby closing the pores which are created during the initial release phase (due to the release of active agent from the surface). Therefore, a plasticizer is added to a nanoparticle formulation disclosed herein to maintain the glass transition temperature above 37° C. despite a decline in molecular weight of the polymer with time. In this manner, the pores remain open and facilitate a continuous release of the encapsulated active agent. Suitable plasticizers are generally inert and can be food/medical grade or non-toxic plasticizers including, but not limited to, triethyl citrate (e.g. Citroflex®, Morflex Inc., Greensboro, N.C.), glyceryl triacetate (e.g, Triacetin, Eastman Chemical Company, Kingsport, Tenn.), L-tartaric acid dimethyl ester (i.e. dimethyl tartrate, DMT) and the like. A particularly suitable plasticizer is L-tartaric acid dimethyl ester.

The amount of plasticizer employed in a nanoparticle composition can range from about 5 to 40 weight percent of the nanoparticle, more desirably from about 10 to 20 weight percent of the nanoparticle. In particular embodiments, the plasticizer encompasses about 10 weight percent of the nanoparticle composition.

By enhancing the release profile of an active agent, a plasticizer-containing nanoparticle has utility in the delivery of telomerase (its subunits or variants) to a variety of tissues or organs. Accordingly, the present invention further relates to a composition for sustained or continuous release of an effective amount of telomerase, wherein said composition contains telomerase, at least one biodegradable polymer, and a plasticizer. As used herein, controlled release, sustained release, or similar terms are used to denote a mode of active agent delivery that occurs when the active agent is released from the nanoparticle formulation at an ascertainable and controllable rate over a period of time, rather than dispersed immediately upon application or injection. Controlled or sustained release can extend for hours, days or months, and can vary as a function of numerous factors. For the composition of the present invention, the rate of release will depend on the type of the plasticizer selected and the concentration of the plasticizer in the composition. Another determinant of the rate of release is the rate of hydrolysis of the linkages between and within the polymers of the nanoparticle. Other factors determining the rate of release of an active agent from the present composition include particle size, acidity of the medium (either internal or external to the matrix) and physical and chemical properties of the agent in the matrix.

A sustained release nanoparticle formulation containing an optional plasticizer can be used to deliver telomerase, hTert, hTR, or variants of either, in an amount which is sufficient to effect prevention or treatment of a disease or condition in a subject. Because telomerase has a short cellular lifespan a regulated and sustained release of the enzyme may be critical in maximizing the therapeutic affect over time. This includes administration of the telomerase-loaded nanoparticles to a subject according to standard methods of therapeutic delivery (e.g, topical, intralesional, injection, such as subcutaneous, intradermal, intramuscular, intraocular, or intra-articular injection, and the like).

The nanoparticle may be structurally coated with telomere-associated moieties. Certain telomerase associated proteins are known to cause the localization of telomerase to the telomere end. The telomerase associated proteins can be covalently attached to the surface or in a layer within the nanoparticle (or in the core). These proteins may improve the accessibility of telomerase to the ends of the telomere and or improve its efficacy by more efficiently localizing the nanoparticle near the telomere ends. In U.S. Patent Application 20070020722 the telomere protein known as hPot1 was fused to hTert. This construct has been shown to elongate chromosomal telomeres in a much more rapid fashion due to faster telomerase localization near the telomere end. Coating the surface of a nanoparticle with hPot1 may be expected to direct the nanoparticle to the end of the telomere where telomerase can be released in the vicinity of the telomere end resulting in superior telomere elongation. hTRF2 is also noted for its ability to direct telomerase to telomere ends (Autexier et al., Annu. Rev. Biochem 2006; 75:493-517). This localization concept may be useful in normal cells to enhance the elongation effect. In certain cell types, such a co-delivery strategy may be particularly needed due to insufficient supply of telomere-associated proteins that can chaperone telomerase to the telomere end. Other telomere associated proteins that may aid with telomere accessibility or localization and that are known to localize in the nucleus (and may also prevent deportation of the enzyme elsewhere) or at telomere ends that may also be useful to administer on the nanoparticle surface or in a layer include p43 (Möllenbeck et al., Journal of Cell Science 2003; 116, 1757-1761), hsp90, p23, p80, p95, 14-3-3 proteins, hnRNPs C1, C2, A1 and UP1, (Microbiol Mol Biol Rev. 2002; 66(3):407-425), p23, chaperone, TEP1, 14-3-3, c-Abl, Ku, hESTI, KIP, PinX1, and MKRN1. hTRF2, hPot1 (Autexier et al., Annu. Rev. Biochem 2006; 75:493-517).

As will be appreciated by the skilled artisan, the nanoparticle compositions of the present invention can further contain additional fillers, excipients, binders and the like depending on, e.g., the route of administration and purpose of the nanoparticle such as in cosmetics or as a food substance for example. A generally recognized compendium of such ingredients and methods for using the same is Remington: The Science and Practice of Pharmacy, Alfonso R. Gennaro, editor, 20th ed. Lippingcott Williams & Wilkins: Philadelphia, Pa., 2000.

The nanoparticle may have tolerances (from 0.01% to 40%) for combinations of certain “static” biodegradable biocompatible materials that might alter the nanoparticle's release properties or that may be added to the formulation simply to cause variation. The reason that these polymers are defined as “static” is due to the fact that their incorporation into the formula might be expected to have a net “neutral” effect that neither greatly subtracts or enhances the nanoparticle's properties as described herein. The polymers listed have certain similar properties and so there is a probable tolerance for the substitution of one polymer for another. Some of these polymers may be expected to add plasticity values to the nanoparticle which may affect the sustainability and control of the release of the telomerase protein. Some of those polymers include: Acrylonitrile-Butadiene-Styrene (ABS), Allyl Resin (Allyl), polycondensate, Cellulosic, Epoxy, polyadduct, Ethylene vinyl alcohol (E/VAL), Fluoroplastics (PTFE, alongside with FEP, PFA, CTFE, ECTFE, ETFE), Ionomer, Liquid Crystal Polymer (LCP), Melamine formaldehyde (MF), polycondensate, Phenol-formaldehyde (PF), (Phenolic), Polyacetal (Acetal), Polyacrylates (Acrylic), Polyacrylonitrile (PAN), Acrylonitrile, Polyamide (PA), Nylon, Polyamide-imide (PAI), polycondensate, Polyaryletherketone (PAEK), Ketone, Polybutadiene (PBD), Polybutylene (PB), Polycarbonate (PC), polycondensate, Polydicyclopentadiene (PDCP), Polyektone (PK), Polyester, Polyetheretherketone (PEEK), Polyetherimide (PEI), Polyethersulfone (PES), Polyethylene (PE), Polyethylenechlorinates (PEC), Polyimide (PI), Polymethylpentene (PMP), Polyphenylene Oxide (PPO), Polyphenylene Sulfide (PPS), Polyphthalamide (PTA), Polypropylene (PP), Polystyrene (PS), Polysulfone (PSU), Polyurethane (PU), Polyvinylchloride (PVC), Polyvinylidene Chloride (PVDC), or Silicone (SI). A wide variety of ingredients can be added to the nanoparticle, but the core active ingredients are irreplaceable.

A nanoparticle formulation of the present invention can contain certain “static” biodegradable biocompatible materials based on amino acids that may be used in the formulation to potentially enhance some aspect of the nanoparticles delivery, or that may be added to the formulation to cause variation. Such polymers may be derived from the following amino acids: Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine, Valine or a combination thereof. In particular polymers such as poly (lysine), poly(argnine), and poly(histidine) may be desirable since they are basic and hydrophilic by nature.

Other “static” biodegradable biocompatible materials might be used in the formulation in small amounts either as plasticizers or as ingredients with satisfactory biocompatibility to permit formulation variation. Some of those materials include Polystyrenes of all kind, Poly(styrene-co-chloromethylsytrene), Poly(styrene-co-chloromethylstyrene-co-methyl-4-vinylbenzyl)ether, Poly(styrene-co-chloromethylsytrene), Polyphosphoester, Poly[1,4-bis(hydroxyethyl) terephthalate-co-ethyloxyphosphate]. Polyphosphazenes, Poly(bis(4-carboxyphenoxy) phosphazene), Poly(bis(4-carboxyphenoxy)phosphazene), Poly(bis(1-(ethoxycarbonyl)methylamino)phosphazene), Poly(bis(1-(ethoxycarbonyl)-2-phenylethylphosphazene, Aliphatic Polyesters, Poly(1,4-butylene adipate-co-polycaprolactam), Polycaprolactone, Polycaprolactone, Polyglycolide, Poly(DL-lactide), Poly(DL-lactide-co-caprolactone), Poly(DL-lactide-co-caprolactone), Poly(L-lactide-co-caprolactone-co-glycolide), Poly(DL-lactide-co-glycolide), Poly(DL-lactide-co-glycolide), PHB PHV & Copolymers such as Poly[(R)-3-hydroxybutyric acid], Poly(3-hydroxybutyric acid-co-3-hydroxyvaleric acid), Poly(3-hydroxybutyric acid-co-3-hydroxyvaleric acid), Poly(1,4-butylene succinate), Nylon 6 pellets, Acrylamidomethyl)cellulose acetate butyrate, (Acrylamidomethyl) cellulose acetate propionate, Starch-13C from algae, Cellulose acetate, Cellulose acetate butyrate, Cellulose acetate phthalate, Cellulose acetate propionate, Cellulose acetate trimellitate, Cellulose, cyanoethylated, Cellulose nitrate, Cellulose nitrate, ASTM D, IPA, Cellulose nitrate, ASTM Cellulose colloidal, Cellulose microcrystalline, Cellulose microcrystalline, Cellulose propionate, Cellulose triacetate, Chitosan, Chitosan oligosaccharide lactate, Dextrin palmitate, Ethyl cellulose, 2-Hydroxyethyl cellulose, Hydroxyethylcellulose ethoxylate, 2-Hydroxyethyl cellulose, hydrophobically modified, 2-Hydroxyethyl starch, Hydroxypropyl cellulose, (Hydroxypropyl)methyl cellulose, Hydroxypropyl methyl cellulose phthalate, Hydroxypropyl methyl cellulose phthalate, Maltodextrin, Methyl cellulose, Methyl 2-hydroxyethyl cellulose, Sodium carboxymethyl cellulose, Agar, Alginic acid Sodium salt, PEG Based Polymers, Poly(ethylene glycol)-block-polylactide methyl ether PEG, Di[poly(ethylene glycol)]adipate, Di[poly(ethylene glycol)]adipate, Hexaethylene glycol, Pentaethylene glycol, Polyethylene-block-poly(ethylene glycol), Poly(ethylene glycol), Poly(ethylene glycol) dibenzoate, Poly(ethylene glycol) bis(carboxymethyl)ether, Poly(ethylene glycol) butyl ether, Poly(ethylene glycol) diacrylate, Poly(ethylene glycol) dimethacrylate, Polyethylene glycol dimethyl ether, Polyethylene glycol distearate, Poly(ethylene glycol) divinyl ether, Poly(ethylene glycol) ethyl ether methacrylate, Poly(ethylene glycol) 2-[ethyl[(heptadecafluorooctyl)sulfonyl]amino]ethyl ether, Poly(ethylene glycol) 2-[ethyl[(heptadecafluorooctyl)sulfonyl]amino]ethyl methyl ether, Poly(ethylene glycol), α-maleimidopropionamide-ω-formyl, Poly(ethylene glycol) methacrylate, Poly(ethylene glycol) methyl ether, Poly(ethylene glycol)-block-poly(ε-caprolactone)methyl ether PEG, polycaprolactone, Poly(ethylene glycol)-block-poly(lactone) methyl ether PEG, polylactide, Poly(ethylene glycol) methyl ether methacrylate, Poly(ethylene glycol) myristyl tallow ether, Poly(ethylene glycol) 4-nonylphenyl ether acrylate, Poly(ethylene glycol) phenyl ether acrylate, Poly(ethylene glycol) phenyl ether acrylate, Poly(ethylene glycol) phenyl ether acrylate, Poly(ethylene glycol), reacted with Bisphenol A diglycidyl ether, Poly(ethylene glycol) tetrahydrofurfuryl ether, Poly(ethylene oxide), Poly(ethylene oxide)-block-polylactide, Poly(ethylene oxide), four-arm, amine terminated, Poly(ethylene oxide), four-arm, carboxylic acid terminated, Poly(ethylene oxide), four-arm, hydroxy terminated, Poly(ethylene oxide), four arm, succinimidyl glutarate terminated, Poly(ethylene oxide), four-arm, succinimidyl succinate terminated, Poly(ethylene oxide), four-arm, thiol terminated, Poly(ethylene oxide), six-arm, hydroxyl, Tetraethylene glycol dimethyl ether, Poly(ethylene glycol)-block-polylactide methyl ether PEG, Polylactide-block-poly(ethylene glycol-block-polylactide PLA average Mn 2000, PEG average Mn 1000, Poly(ethylene glycol) di-(4-hydroxyphenyl)diphenylphosphine loading: 0.5-1.0 mmol/g P PEG-PPG Copolymers, Poly(ethylene glycol)-block-poly(propylene glycol)-block-poly(ethylene glycol), Poly(ethylene glycol-ran-propylene glycol), Poly(ethylene glycol-ran-propylene glycol) monobutyl ether, Poly(propylene glycol)-block-poly(ethylene glycol)-block-poly(propylene glycol) bis(2-aminopropyl ether), Poly(propylene glycol)-block-poly(ethylene glycol)-block-poly(propylene glycol) bis(2-aminopropyl ether), Poly(propylene glycol)-block-poly(ethylene glycol)-block-poly(propylene glycol) bis(2-aminopropyl ether),

Polyanhydrides, 1,3-Bis(4-carboxyphenoxy)propane, 1,6-Bis(p-acetoxycarbonylphenoxy) hexane, Poly(sebacic acid), diacetoxy terminated, Poly[1,6-bis(p-carboxyphenoxy)hexane, Poly[(1,6-bis(p-carboxyphenoxy)hexane)-co-sebacic acid], Poly(vinyl alcohol), Poly(vinyl alcohol-co-ethylene) ethylene, Poly(vinyl alcohol-co-vinyl acetate-co-itaconic acid), Poly(vinyl chloride-co-vinyl acetate-co-vinyl alcohol).

Like plastic polymers dendrimers might be used in the formulation to add values to the rigidity of the nanoparticle. Such dendrimers include: PAMAM Dendrimer, 1,4-diaminobutane core, PAMAM Dendrimer, 1,12-diaminododecane core, PAMAM Dendrimer, 1,6-diaminohexane core, PAMAM Dendrimers of all kind, PAMAM-OH Dendrimer, PAMAM-OS-trimethoxysilyl dendrimer, PAMAM-OS-trimethoxysilyl dendrimer, PAMAM-OS-trimethoxysilyl dendrimer, PAMAM-OS-trimethoxysilyl dendrimer, PAMAM-OS-trimethoxysilyl dendrimer, PAMAM-OS-trimethoxysilyl dendrimer, PAMAM-succinamic acid dendrimer, PAMAM-amidoethyl ethanolamine dendrimer, PAMAM-hexylamide dendrimer, PAMAM-hexylamide dendrimer, PAMAM-tris(hydroxymethyl)amidomethane, PAMAM-tris(hydroxymethyl) amidomethane dendrimer, Phosphonitrilic chloride trimer, Cyclotriphosphazene-PMMH-6 Dendrimer, Cyclotriphosphazene-PMMH-12 Dendrimer, Cyclotriphosphazene-PMMH-24 Dendrimer, Cyclotriphosphazene-PMMH-48 Dendrimer, Cyclotriphosphazene-PMMH-96 Dendrimer, Cyclotriphosphazene-PMMH-192 Dendrimer, Cyclotriphosphazene-PMMH-6 Dendrimer, Cyclotriphosphazene-PMMH-24 Dendrimer, Cyclotriphosphazene-PMMH-48 Dendrimer, Cyclotriphosphazene-PMMH-96 Dendrimer, Thiophosphoryl chloride, Thiophosphoryl-PMMH-3 Dendrimer, Thiophosphoryl-PMMH-6 Dendrimer, Thiophosphoryl-PMMH-12 Dendrimer, Thiophosphoryl-PMMH-24 Dendrimer, Thiophosphoryl-PMMH-48 Dendrimer, Thiophosphoryl-PMMH-96 Dendrimer, Thiophosphoryl-PMMH-3 Dendrimer, Thiophosphoryl-PMMH-6 Dendrimer, Thiophosphoryl-PMMH-12 Dendrimer, Thiophosphoryl-PMMH-24 Dendrimer, Thiophosphoryl-PMMH-48 Dendrimer, DAB-Am-4, Polypropylenimine tetraamine Dendrimer, DAB-Am-8, Polypropyl-enimine octaamine Dendrimer, DAB-Am-16, Polypropylenimine hexadecaamine Dendrimer, DAB-Am-32, Polypropylenimine dotriacontaamine Dendrimer, DAB-Am-64, Polypropyl-enimine tetrahexacontaamine Dendrimer.

Hydrogels may also be incorporated because they may contain favorable biocompatible biodegradable features or they may be incorporated in order to change application features; making it possible for example to contain nanoparticles in a part of the body on a stent or in some highly localized therapy where it is important for the nanoparticle not to greatly circulate in the blood stream. These hydrogels include Poly(acrylic acid-co-acrylamide), Potassium salt cross-linked, Poly(2-hydroxyethyl methacrylate), Poly(2-hydroxyethyl methacrylate), Poly(2-hydroxyethyl methacrylate), Poly(acrylic acid), Poly(isobutylene-co-maleic acid), Poly(isobutylene-co-maleic acid), Poly(N-isopropylacrylamide), Lignosulfonic acid Sodium, Polyacrylamide, Poly(acrylamide-co-acrylic acid), Poly(acrylic acid), Poly(acrylic acid-co-maleic acid), Poly(acrylic acid-co-maleic acid), Poly(acrylic acid-co-maleic acid), Poly(acrylic acid), Poly(acrylic acid, sodium salt), Poly(acrylonitrile-co-butadiene-co-acrylic acid), Poly(allylamine), Poly(ethylene-co-acrylic acid), Poly(ethylene-co-methyl acrylate-co-acrylic acid), Poly(ethylene-co-methyl acrylate-co-acrylic acid), Poly[(isobutylene-alt-maleic acid, ammonium salt)-co-(isobutylene-alt-maleic anhydride)], Poly[(isobutylene-alt-maleic acid, ammonium salt)-co-(isobutylene-alt-maleic anhydride)], Poly(isobutylene-alt-maleic anhydride), Poly(isobutylene-alt-maleic anhydride), Poly(isobutylene-alt-maleic anhydride), Poly[(isobutylene-alt-maleimide)-co-(isobutylene-alt-maleic anhydride)], Poly[(isobutylene-alt-maleimide)-co-(isobutylene-alt-maleic anhydride)], Poly(methyl vinyl ether-alt-maleic anhydride), Poly(propylene glycol), Poly(vinyl acetate), Poly(vinyl butyral-co-vinyl alcohol-co-vinyl acetate), Poly(4-vinylpyridine), Polyvinylpyrrolidone, Cucurbit, Polyacrylonitrile, Poly(1-decene-sulfone), Poly(1-dodecene-sulfone), Poly(2-ethylacrylic acid), Poly(ethylene terephthalate), Poly(ethylene terephthalate), Poly(ethylene terephthalate), Poly(1-hexadecene-sulfone), Poly(hexafluoropropylene oxide), Poly(hexafluoropropylene oxide), Poly(1-hexene-sulfone), Poly(methyl vinyl ether), Poly(1-octene-sulfone), Poly(perfluoropropylene oxide-co-perfluoroformaldehyde), Poly(perfluoropropylene oxide-co-perfluoroformaldehyde), Poly(perfluoropropylene oxide-co-perfluoroformaldehyde), Poly(2-propylacrylic acid), Poly(propylene glycol) bis(2-aminopropyl ether), Poly(propylene glycol) bis(2-aminopropyl ether), Poly(propylene glycol) bis(2-aminopropyl ether), Poly(propylene glycol) diglycidyl ether, Poly(propylene glycol) diglycidyl ether, Poly(propylene glycol) methacrylate, Poly(propylene glycol) methyl ether acrylate, tripropylene glycol, Poly(propylene glycol) monobutyl ether, Poly(propylene glycol) monobutyl ether, Poly(propylene glycol) 4-nonylphenyl ether acrylate average, Poly(propylene glycol), tolylene 2,4-diisocyanate, Poly(propylene glycol), tolylene 2,4-diisocyanate terminated, Poly(1-tetradecene-sulfone), Poly(tetrahydrofuran), Poly(vinylbenzyl chloride),60/40 mixture of 3- and 4-isomers, Poly(vinylidene fluoride), Poly(4-vinylphenol), Poly(4-vinylpyridine-co-styrene), 4-Bis(acryloyl)piperazine, 1,4-Cyclohexanedimethanol divinyl ether, Di(ethylene glycol) diacrylate, Di(ethylene glycol) dimethacrylate, N,N′-(1,2-Dihydroxyethylene)bisacrylamide, Divinylbenzene, p-Divinylbenzene, Ethylene glycol diacrylate, Ethylene glycol dimethacrylate, 1,6-Hexanediol diacrylate technical grade, 4,4′-Methylenebis(cyclohexyl isocyanate), 1,4-Phenylenediacryloyl chloride, Tetra(ethylene glycol) diacrylate, Triethylene glycol dimethacrylate

Telomerase is composed of hTert and hTR. Variants of telomerase which can be formulated in a nanoparticle of the present invention to treat a disease or condition of aging include the full length isotype of telomerase derived from wild-type hTert, telomerase derived from a “nuclear only” variant (Santos et al. Aging Cell 2004 6: 399-411), telomerase derived from a stabilized form of hTert known as hTrt.sup.plus, which was deposited in the DSMZ (German Collection of Microorganisms and Cell Cultures) (DSM 14569) in accordance with the Budapest treaty on 17 Oct. 2001, and other variants derived from species of hTert described herein.

The wild-type human hTert cDNA sequence can be acquired from the GenBank with the Locus ID of NM_{—}198253; the wild-type human hTert amino acid sequence can be acquired from the GenBank with the Locus ID of NP_{—}937983. Both sequences can be viewed and verified from the following web-link:

http://www.ncbi.nlm.nih.gov/sites/entrez?db=Nucleotide

A variant of hTert known as hTRT.sup.plus contains additional introns which is alleged to stabilize the enzyme. “Nuclear only” hTert (nuclear hTert) is similar to the wild-type hTert enzyme with an important distinction that makes it novel. Nuclear hTert stays confined to the nucleus of a cell and thereby reduces the amount of apoptosis correlated with a frequent problem observed in wild-type telomerase, which is its leakage into the mitochondrial compartment. The amino acid distinctions between nuclear Tert and wild-type Tert are described in detail the following study (Santos et al, Aging Cell 2004; 6:399-411). Wild-type hTert has a tendency to slow cell growth and result in apoptosis while on the other hand nuclear Tert might be expected to lead to safer and more powerful therapeutics since cell cultures grown with nuclear hTert grow more rapidly. Nuclear hTert or telomerase derived from nuclear hTert (nuclear telomerase) can be delivered with a biodegradable nanoparticle using the formulation, compositions, and methods described herein.

TABLE 2
hTert variants that may also be delivered to improve function of hTert in vivo

| Key | From | To | Length | Description | FTId |
|---|---|---|---|---|---|
| CHAIN | 1 | 1132 | 1132 | Telomerase reverse transcriptase. | PRO_0000054925 |
| DOMAIN | 605 | 935 | 331 | Reverse transcriptase. | |
| MOD_RES | 1113 | 1113 | | Phosphothreonine. | |
| MOD_RES | 1125 | 1125 | | Phosphoserine. | |
| VAR_SEQ | 764 | 807 | | STLTDLQPYMRQFVAHLQETSP | VSP_019587 |
| | | | | LRDAVVIEQSSSLNEASSGLFD−> | |
| | | | | LRPVPGDPAGLHPLHAALQPVL | |
| | | | | RRHGEQAVCGDSAGRAA | |
| | | | | PAFGG (in isoform 2). | |
| VAR_SEQ | 808 | 1132 | | Missing (in isoform 2). | VSP_019588 |
| VAR_SEQ | 885 | 947 | | Missing (in isoform 3). | VSP_021727 |
| VARIANT | 412 | 412 | 1 | H −> Y. | VAR_025149 |
| VARIANT | 1062 | 1062 | 1 | A −> T. | VAR_025150 |
| MUTAGEN | 712 | 712 | | D−>A: Loss of telomerase activity. | |
| MUTAGEN | 868 | 869 | | DD−>AA: Loss of telomerase | |
| | | | | activity. | |
| MUTAGEN | 868 | 868 | | D−>A: Loss of telomerase activity. | |
| MUTAGEN | 869 | 869 | | D−>A: Loss of telomerase activity. | |
| CONFLICT | 516 | 516 | | D −> G (in Ref. 2). | |

Variants that are not known to improve the function of hTert can also be effectively delivered with this technique. These variants might be delivered with the methods and compositions described herein or used to reconstitute telomerase that might be delivered include those in Table 2.

hPot-hTert or hPot-hTert.sub.₊₁₂₈ are described in U.S. Patent Application 20070020722. In this variant the telomerase-associated protein hPot1 is bound to the telomerase enzyme to increase its “proximity effectiveness” by localizing it closer to the telomere ends. We postulate hPot1 could be attached to nuclear hTert to form either nuclear hPot-hTert or nuclear hPot-hTert.sub.₊₁₂₈, which might represent a more efficacious form of telomerase.

An effective amount of telomerase or hTert present in a nanoparticle formulation of the present invention is an amount which may address a disease or condition of aging. Certain diseases of aging are noted to be caused by or correlate closely with telomere shortening for a variety of reasons such as cellular senescence, changes in protein output, metabolic disruption, and other programmatic changes that occur in a cell as the telomere shortens (Funk et al., Experimental Cell Research, 2000; 258(2): 270-278). Any disease that is caused by telomere shortening might be potentially treated or reduced by reversing this shortening. Critically short telomeres are present in many of the most common degenerative diseases (Harley, Current Molecular Medicine 2005; 5(2):205-11). The restoration of wild-type telomere lengths represents a possibly novel way to reverse or lessen the effects of aging in these diseases. Some of the most common telomere-associated diseases include idiopathic pulmonary fibrosis, dyskeratosis congenita, aplastic anemia, arteriosclerosis, cirrhosis of the liver and kidney, osteoporosis, arthritis, Alzheimer's, type 2 diabetes, macular degeneration, age-related immune dysfunction that may be virally induced (Elaine Shmidt, UCLA Health & Medicine News, Nov. 12, 2004), Myelodysplastic Syndrome (Moffitt, Cancer Control 11(2):77-85, 2004), Dsykeratosis (Blanche P. Alter, MD, MPH, May 14th at the 2005 Pediatric Academic Societies' Annual Meeting), in addition to others. It has also been noted that telomerase plays an important role in wound healing. Based on this premise the therapeutic administration of telomerase might improve wound healing capacity (Jiang et al., United States Patent Application 20060239974). It is contemplated that a telomerase-containing nanoparticle formulation of the present invention can be administered via intravenous, intracerebral, intracarotid, intramuscular or intrajugular routes, wherein intracarotid or intrajugular routes are suitable. The exact amount of telomerase to be administered will vary according to factors such as the tissues being targeted as well as the other ingredients in the composition. The effectiveness of the treatment can be determined by monitoring visible signs of aging, response of disease states to the therapy, or very precise measurements of certain proteins such as EPC1 in connective tissues (cartilage) of a mammal (Lanza et al., Science 2000; 288(5466):665-669) or collagen levels in the skin (Bodnar et al., Science 1998; 279(5349):349-352).

It is postulated that telomerase loaded nanoparticles may be useful in the treatment of certain neurological diseases of aging such as Alzheimer's and Parkinson's (Mattson et al., J Mol. Neurosci. 2000; 14(3):175-82). In studies performed by Labhasetwar and colleagues, PLGA nanoparticles were employed in an in vivo treatment model of post stroke reperfusion injury wherein delivery of the active agent was targeted to the brain (Labhasetwar et al., U.S. Pat. No. 7,332,159). While neuronal tissues are not dividing in nature there is evidence that telomerase can regenerate and cause the division of progenitor cells and stem cells (Zimmerman et al., Stem Cells 2004; 22:741-749) that can reconstitute the health of the brain organ.

It is envisioned that the administration of telomerase via nanoparticles may stabilize premalignant tissues and prevent the development of certain forms of cancer, such as leukemia. Defects of telomerase and critical telomere shortening are noted in Myelodisplastic Syndrome (a precursor to leukemia) (Moffit, Cancer Control 2004; 11(2):77-85), for example, while critically short telomeres are observed in a high percentage of pre-malignant tissues (potentially 96%) (Meeker et al., Clinical Cancer Research 2004; 10: 3317-3326). The administration of telomerase based therapeutics may help prevent the onset of certain forms of neoplasia that associate closely with critical telomere shortening.

The invention herein is envisioned to aid scientists in the growth of tissues outside of the body. Skin cells can only replicate a limited number of times in the production of skin grafts (Geoffrey Mock, Apr. 22, 2003, Duke University News and Communication). For burn victims who need new skin this represents a challenge as it becomes hard to grow even a few centimeters of artificial skin. This leads to thin and frail skin grafts because telomeres erode quickly during replication. The compositions and methods described herein may make economical the large-scale growth of higher quality skin grafts. Likewise improved methods of growing cell cultures for study, or transplantation may become possible since mammalian cells erode their telomeres quickly in in vitro study conditions. Similarly laboratories can potentially produce larger volumes of human peptides and proteins if the lifespan of cell culture materials can be extended. Similar to the growth of skin, the growth of artificial organs encounters growth obstacles in the laboratory as a result of telomere exhaustion. Various kinds of organs and transplantable living tissues such as artificial corneas may benefit from the invention described herein. For a number of reasons understood by those skilled in the art an ex vivo nanoparticle based protein therapy may be safer and more preferred by regulators than a gene therapy or a protein therapy based on a virus.

The invention herein is envisioned to treat aging of the skin as a cosmetic or cosmaceutical product. Age-related atrophy and wrinkling of the skin are among the most common problems of aging populations. Studies demonstrate that these afflictions correlate closely to telomere shortening (Blasco, Nat Rev Genet. 2005; 6(8): 611; Leutwyler, Scientific American, Feb. 2, 1998; Serrano et al., Circ Res 2004; 94:575-584). Telomerase loaded nanoparticles could be combined in a variety of cosmetic bases that could be applied to the skin as a cosmetic or cosmaceutical (regulated cosmetic) agent. In this application the skin, particularly fibroblast and keratinocytes of human cells, can become rejuvenated and thereby reduce many of the signs of skin aging such as wrinkling. In the past, skin cells altered to express the hTert gene, which causes a continuous supply of telomerase in a cell, have been shown to produce high levels of elastin and collagen, which are proteins that improve skin's elasticity and sheen (Baur et al., Science 2001; 292(5524):2075-2077). This technique has been used by W. D. Funk to regenerate aged skin in animal models (Funk et al., Experimental Cell Research, 2000; 258(2):270-278). The administration of the raw protein telomerase or hTert might represent a safer and publicly preferred tactic over a gene therapy. Because nanoparticles are water soluble, an oil-based cosmetic may be preferable.

The invention herein is envisioned for use in food stuffs and health food products, such as sports drinks, vitamins, natural food substances, etc. to provide health benefits to the consumer. There is evidence that the restoration of healthful telomere lengths may considerably regenerate the cells of a patient (Harley, Current Molecular Medicine 2005; 5(2):205-211). Effectively all people suffer from aging in some form. Recent studies indicate that telomere length plays a significant role in the longevity of humans (Cawthon, The Lancet 2003; 361(9355): 393-395), while it is known that younger individuals tend to have better health than older individuals.

By way of illustration, in this study telomerase was successfully entrapped into nanoparticles and delivered to human fibroblast tissues. Telomere elongation was noted and documented.

### EXAMPLES AND FORMULATIONS

### Example 1

In Example 1, both active telomerase and active hTert were delivered to fibroblast cells in a biodegradable nanoparticle. Recombinant telomerase and recombinant hTert were obtained from Advanced Product Enterprises LLC (Frederick, Md., USA). Both proteins were in solution containing 10% glycerol in 1× CHAPS buffer and total protein concentrations were greater than 10 mg/ml. The use of “cryoprotectant” agent like glycerol is imperative to preserve the activity of the enzyme so that it survives a freeze thaw cycle or can otherwise be stored. Quantitative telomerase detection Kit from US Biomax, Inc (Rockville, Md., USA) was used to measure telomerase activities of recombinant active and inactive hTert. Recombinant telomerase and hTert were diluted to 10, 100 and 400 folds, 1 μl each diluted and 1 μl recombinant protein without dilution were included in the telomerase activity detection assay. Significant telomerase activity was detected for active telomerase. In other literature the introduction of hTert without hTR appears to elongate telomeres by reconstituting the whole enzyme in vivo; this process appears to be very slow, so a longer endpoint might be expected to yield better results when examining the delivery of the hTert protein alone.

PLGA nanoparticles containing recombinant telomerase or hTert was formulated by using a novel double emulsion-solvent evaporation technique previously optimized by Prabha and Labhasetwar (2). In a typical preparation, a solution of recombinant active or inactive hTert (200 μl, approximately 2 mg) and acetylated bovine serum albumin (BSA) (2 mg) were added to a solution of polymer (30 mg/l ml chloroform PLGA 50:50, inherent viscosity 1.32 dl/g; Lactel, Ala., USA) and emulsified with a probe sonicator (3 W/2 min. over ice; Sonicator XL, Misonix, Farmingdale, N.Y., USA). The primary emulsion were then added to 6 mL of a poly-vinyl alcohol (PVA) solution (2% w/v in Tris-EDTA), vortexed to form the double emulsion, and sonicated (3 W/5 min. over ice). The double emulsion was then stirred in a chemical fume hood for 18 h at room temperature followed by stirring for 1 h under vacuum to evaporate chloroform. PLGA nanoparticle thus formed were recovered by ultracentrifugation (100,000 RCF, 20 min at 4° C., WX80 ultracentrifuge with T-865 rotor, Thermo, Mass., USA), washed twice to remove PVA and unentrapped recombinant telomerase or hTert by resuspending nanoparticle pellet in 5 mL Tris-EDTA and centrifuging as previously described. The final nanoparticle pellet were resuspended in 2 mL sterile, nuclease-free dH₂O, aliquotted into pre-weighed, sterile tubes, frozen (−80° C.), lyophilized to obtain a dry powder, and weighed. A BSA only control nanoparticle formulation with 2 mg acetylated bovine serum in 200 μl Tris-EDTA was also prepared using the same method. All of the previous supernatants were reserved for the determination of recombinant telomerase or hTert and BSA entrapment efficiency.

Fibroblast cells (ATCC, SCRC-1041) to be transfected were seeded in four 6-well plates at a density of 1.4×10⁵ cells/well in complete growth medium (containing 15% v/v serum). Kept n=3 wells for each sample of nanoparticle in one 6-well plate. Cells at the same density were seeded for cell only control wells to which no nanoparticle would be added. Allowed the cells to attach and grow in the plates for 24 h. Used sterile conditions to prepare stock suspensions of active telomerase-, active hTert- and BSA-loaded nanoparticle (9 mg in 1.1 ml of DMEM serum-free medium). nanoparticle suspensions were sonicated in a water bath sonicator for 10 min. Each nanoparticle suspensions were diluted to 24 ml with complete medium. Aspirated the medium from the wells and added 4 ml of nanoparticle suspension to each well. Two 6-well plates with three wells each of active telomerase, active hTert and BSA only nanoparticle and cell only controls were incubated for 24 h at 37° C. in 5% CO₂, the other two 6-well plates with the same samples were incubated for 48 h.

After 24 h or 48 h incubations, cells transfected with different nanoparticle preparations and cells in cell only wells were washed twice with PBS (pH 7.4) and harvested by scrapping with a rubber policeman. Genomic DNA was isolated using DNeasy blood & tissue kit (Qiagen, Chatsworth, Calif., USA) from each well and DNA concentration was measured with picogreen assay (Invitrogen, Carlsbad, Calif., USA). Genomic DNA samples were diluted to 5 ng/μl, and 25 ng of DNA was added in each well of a 96-well plate and air-dried.

Quantitative PCR reaction was used to measure relative average telomere length (2, 3). The telomere repeat copy number to single gene copy number (T/S) ratio was determined using Biorad (Hercules, Calif., USA) iQ5 real-time PCR detection system in a 96-well format. The telomere reaction mixture consisted of 1× Qiagen Quantitect Sybr Green Master Mix, 2.5 mmol/L of DTT, 100 nmol/L of Tel-1b primer (CGGTTTGTTTGGGTTTGGGTTTGGGTTTGGGTTTGGGTT), and 900 nmol/L of Tel-2b primer (GGCTTGCCTTACCCTTACCCTTACCCTTACCCTTACCCT). The reaction proceeded for 1 cycle at 95° C. for 3 min, followed by 40 cycles at 95° C. for 15 s, and 54° C. for 1 min. The β-globin reaction consisted of 1× Qiagen Quantitect Sybr Green Master Mix, 300 nmol/L of hbg1 primer (GCTTCTGACACAACTGTGTTCACTAGC), and 700 nmol/L of hbg2 primer (CACCAACTTCATCCACGTTCACC). The β-globin reaction proceeded for 1 cycle at 95° C. for 3 min, followed by 40 cycles at 95° C. for 15 s, 58° C. for 20 s, and 72° C. for 28 s. All samples for both the telomere and single-copy gene (human β-globin) reactions were done in triplicate. In addition to the samples, each 96-well plate contained a six-point standard curve from 5 to 100 ng using human genomic DNA (Promega, Madison, Wis., USA).

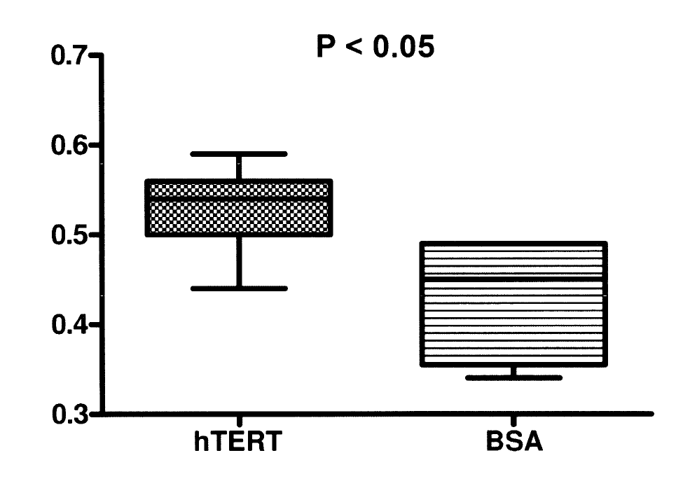

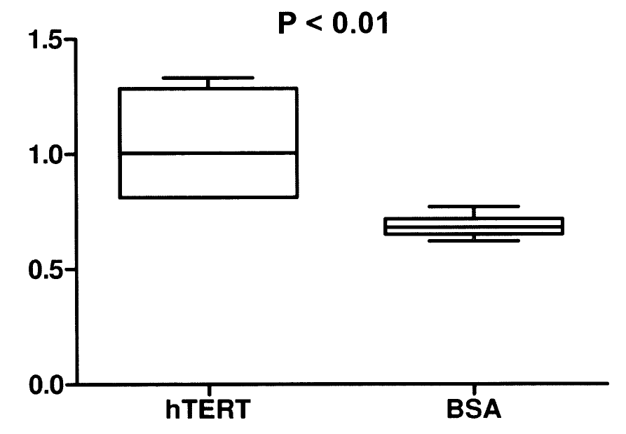

Using two-tailed student's t-test, we demonstrated that there is statistically significant elongation of cells transfected with active telomerase when compared to BSA only controls after 24 h. The relative telomere length for active telomerase had an average (±SE) of 0.53±0.02 while BSA only control had an average (±SE) of 0.43±0.03 (FIG. 1). After 48 h incubation, we observed even more pronounced telomere elongation in cells transfected with active telomerase. The relative telomere length for active telomerase had an average (±SE) of 1.04±0.1 while BSA only control had an average (±SE) of 0.69±0.02 (FIG. 2). When compared to BSA only controls active hTert appeared to have minimal activity in fibroblast cells over the time period studied, however, previous literature seems to indicate that hTert alone may be expected to elongate telomeres efficiently in other cell types or possibly over a longer study period.

It has been known that the delivery of hTert alone causes the reconstitution of human telomerase in some cell lines. Therefore, since hTert is more stable than its holoenzyme, stimulation of the holoenzyme by its hTert subunit has been a preferred method for studying telomerase in vitro. The exogenous delivery of the whole enzyme telomerase, which is nearly twice the size of its hTert subunit and includes a delicate RNA component, has not been mentioned in previous literature. The encapsulation of proteins in nanoparticles can be a violent process which degrades RNA. Additionally, it was not known or reported whether the RNA component of telomerase would survive the encapsulation procedure. Surprisingly, this study demonstrates that in fibroblasts the delivery of the full telomerase enzyme leads to much more rapid and more therapeutic elongation of telomeres than hTert protein alone (which was essentially non-proccessive). The use of telomerase rather than hTert is novel in this case and is for the first time demonstrated as possible. There may be reasons to favor the delivery of the whole enzyme telomerase over its catalytic subunit for some applications. It may, for example, be important to consider that some cell lines may lack the accessible hTR component that together with hTert forms telomerase, or that they may lack the machinery or tendency to reconstitute whole telomerase since the levels of telomere-associated proteins and the handling and or resistance to hTert appears to vary in different types of cells. On the other hand, the catalytic subunit is easier to store and is a less delicate molecule, and may therefore carry advantages over the holoenzyme in some applications. For these reasons it was important to deliver both enzyme components in a biodegradable nanoparticle.

1. A method for treating a disease of aging comprising administering an effective amount of telomerase, wherein said telomerase, its catalytic subunit, or a known variant of either, is formulated in a nanoparticle and administered orally, via the carotid artery or jugular vein, intravensouly, topically or in other common methods of administration, to a subject in need of treatment, thereby reversing or lessing one or more diseases or conditions of biological (telomere related) aging.

2. The method of claim 1, wherein the whole telomerase enzyme is delivered to cells in a biodegradable nanoparticle.

3. The method of claim 1, wherein the hTert enzyme is delivered to cells in a biodegradable nanoparticle

4. The method of claim 1, wherein “nuclear only” hTert or telomerase is delivered to cells in a biodegradable nanoparticle

5. The composition of claim 1, wherein “nuclear only” hTert is delivered to cells in a biodegradable nanoparticle.

6. The method of claim 1, wherein another known variant of hTert or telomerase is delivered to cells in a biodegradable nanoparticle.

7. The composition of claim 1, wherein another known variant of hTert or telomerase is delivered to cells in a biodegradable nanoparticle.

8. The method of claim 1, wherein the biodegradable polymer comprises a poly(lactide-co-glycolide), poly(lactic acid), poly(alkylene glycol), polybutylcyanoacrylate, poly(methylmeth-acrylate-co-methacrylic acid), poly-allylamine, polyanhydride, polyhydroxybutyric acid, poly-caprolactone, lactide-caprolactone copolymers, polyhydroxybutyrate, polyalkylcyanoacrylates, polyanhydrides, polyorthoester or a combination thereof.

9. The method of claim 1, wherein the nanoparticle further comprises a targeting moiety.

10. The method of claim 1, wherein the nanoparticle further comprises a plasticizer to facilitate sustained release of telomerase, hTert, or a combination thereof.

11. The method of claim 1, wherein the plasticizer comprises L-tartaric acid dimethyl ester, triethyl citrate, or glyceryl triacetate.

12. A composition for sustained release of an effective amount of an active agent said composition comprising telomerase, hTert, or a known variant of either, at least one biodegradable polymer, and a plasticizer.

13. The composition of claim 1, wherein the biodegradable polymer comprises a poly(lactide-co-glycolide), poly(lactic acid), poly(alkylene glycol), polybutylcyanoacrylate, poly(methylmethacrylate-co-methacrylic acid), poly-allylamine, polyanhydride, polyhydroxybutyric acid, polycaprolactone, lactide-caprolactone copolymers, polyhydroxybutyrate, polyalkylcyanoacrylates, polyanhydrides, polyorthoester or a combination thereof.

14. The composition of claim 1, wherein the plasticizer comprises L-tartaric acid dimethyl ester, triethyl citrate, glyceryl triacetate or others mentioned in the claim.

15. The composition of claim 1, wherein the nanoparticle may further comprise a targeting moiety.

16. A method for affecting a sustained release of an effective amount of an active agent comprising administering any composition of claim 1 to a subject thereby affecting a sustained release of an effective amount of the active agent to the subject.

17. A method of claim 1 wherein “static” biodegradable biocompatible polymers are mixed with the core ingredients.

18. A composition of claim 1 wherein “static” biodegradable biocompatible polymers are mixed with the core ingredients.

19. A composition of claim 1 wherein dendrimers are incorporated with the core ingredients.

20. A composition of claim 1 wherein hydrogels are incorporated with the core ingredients.

21. A method of claim 1 wherein the nanoparticle may incorporate telomere associated moeites.

22. A composition of claim 1 wherein the nanoparticle may incorporate telomere associated moeites.
